(19) United States
(12) Patent Application Publication
(10) Pub. No.: US 2021/0353150 A1
(43) Pub. Date: Nov. 18, 2021

(54) ISOLATION DEVICES TO REDUCE CONTAMINATION DURING IMAGING OF PATIENTS

(71) Applicant: The USA, as represented by the Secretary, Department of Health and Human Services, Bethesda, MD (US)

(72) Inventors: Bradford J. Wood, Bethesda, MD (US); Baris Turkbey, Bethesda, MD (US); Sheng Xu, Bethesda, MD (US); Evrim Turkbey, Bethesda, MD (US); Peng An, Bethesda, MD (US); Gianpaolo Carrafiello, Bethesda, MD (US); Robert Suh, Bethesda, MD (US); Hayet Amalou, Bethesda, MD (US); Amel Amalou, Bethesda, MD (US); Robin Therme, Bethesda, MD (US); Brad Dunlap, Bethesda, MD (US); Matt Berger, Bethesda, MD (US); Yash Agrawal, Bethesda, MD (US); Geoff Wagner, Bethesda, MD (US); Kurt Haupt, Bethesda, MD (US); Shane Anderson, Bethesda, MD (US); Anna Maria Lerardi, Bethesda, MD (US)

(21) Appl. No.: 17/323,763

(22) Filed: May 18, 2021

Related U.S. Application Data
(60) Provisional application No. 63/026,502, filed on May 18, 2020.

(51) Int. Cl. A61B 5/00; A61B 5/055; A61M 16/06

(52) U.S. Cl. CPC A61B 5/0046; A61B 5/70; A61M 16/0666; A61B 5/6831; A61B 5/7203; A61B 5/055

(57) ABSTRACT

Systems, devices, and methods for isolating and protecting patients during medical imaging procedures. The devices generally related to disposable personal protective equipment (PPE) that may be used to isolate patients and prevent cross-contamination of infectious diseases during medical imaging.

### RELATED APPLICATIONS

This application claims priority to U.S. Provisional Application No. 63/026,502, filed May 18, 2020, which is incorporated by reference in its entirety.

### FEDERALLY SPONSORED RESEARCH OR DEVELOPMENT

This disclosure is made with government support. The Government has certain rights in the inventions disclosed herein.

### FIELD OF THE INVENTION

The present disclosures relates to devices and methods for isolating and preventing cross contamination of patients having an infectious disease during medical imaging.

### BACKGROUND OF THE INVENTION

The use of chest CT during an infectious disease pandemic may introduce contamination risk to staff and nearby patients during imaging procedures. While advanced staff training, dedicated equipment and hallways, and pre-emptive standardized operating procedures may reduce risk to staff, a single infected patient or breach in technique can have profound implications. There is a clinical need for cost-effective disposable personal protective equipment (“PPE”) for the infected patient's isolation while undergoing medical imaging procedures, including but not limited to CT, MRI, and PET imaging procedures.

### SUMMARY

This disclosure provides a personal protective patient isolation apparatus. The patient isolation apparatus may include a vapor barrier; an air filter incorporated within the vapor barrier; an air inlet traversing the vapor barrier; and a fastening strap configured to attach the vapor barrier to a patient.

In an aspect, the vapor barrier and the fastening strap may be configured to form a patient compartment around the patient. The vapor barrier and the fastening strap may be further configured to provide a hermetic seal. The vapor barrier is dimensioned to substantially envelop the patient.

The patient isolation apparatus may further include a standoff device to hold the vapor barrier away from the patient. In some aspects, the standoff device is attached to the vapor barrier. In other aspects, the standoff device is separate from the vapor barrier and configured to be worn on the patient's head.

The air inlet may be configured to allow gas from a gas source to pass through the vapor barrier. In an aspect, the air inlet is an afferent nozzle with a one-way valve for one-way entry of room air to the inside the vapor barrier. In another aspect, the patient isolation apparatus further includes a port traversing the vapor barrier, where the port is configured to connect to a suction source to create negative pressure within the patient compartment of the vapor barrier.

The air filter may be configured to filter air traversing from an interior space formed by the vapor barrier to an exterior environment. In some aspects, the air filter comprises a N-95 filter, a KN-95 filter, a FFP2 material, a HEPA filter, a blended synthetic fiber material, or spun-bound polypropylene.

The vapor barrier may be made of a polymer. In some aspects, the vapor barrier has reduced artifacts on imaging systems, has reduced signal to noise ratio, and does not influence a radiation dose to the patient compared to standard isolation chambers.

In an aspect, a kit may include the patient isolation apparatus; a standoff device not connected to the vapor barrier; and a nasal cannula.

Further provided herein is a method for preventing the transmission of an infectious disease from a patient having an airborne infectious disease during a medical procedure. The method may include covering the patient in a patient isolation apparatus; performing the medical procedure on the patient; and disposing the patient isolation apparatus.

Also provided herein is a method for obtaining medical images of a patient having an airborne infectious disease. The method may include covering the patient in a patient isolation apparatus; positioning the patient to obtain the medical images; obtaining the medical images of the patient; and disposing the patient isolation apparatus.

The medical images may be obtained using a computer tomography (CT) apparatus, a magnetic resonance imaging (MRI) apparatus, or a positron emission tomography (PET) imaging apparatus.

The method may further include connecting a gas source to the air inlet of the patient isolation apparatus, fitting the patient with a nasal cannula connected to the air inlet, and/or connecting a suction source to a port integrated in the vapor barrier.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1 shows a personal protective patient isolation apparatus in one example.

FIG. 2A shows a patient wearing an example personal protective patient isolation apparatus in an upright position.

FIG. 2B shows a patient wearing an example personal protective patient isolation apparatus laying down and the air inlet being connected to a gas source.

FIG. 3 show a patient wearing an example personal protective patient isolation apparatus with an integrated standoff device.

### DESCRIPTION

This disclosure generally relates to systems, devices, and methods for isolating patients using disposable personal protective equipment. Although described in reference to computer tomography imaging, the systems, devices, and methods disclosed herein may be used with a wide variety of medical imaging systems and apparatuses. Additional features and information can be found in the following description, drawings, figures, images, and other disclosure.

Reference to “one embodiment”, “an embodiment”, or “an example” means that a particular feature, structure, or characteristic described in connection with the embodiment is included in at least one embodiment of the disclosure. The appearances of the phrase “in one embodiment” or “in one example” in various places in the specification are not necessarily all referring to the same embodiment, nor are separate or alternative embodiments mutually exclusive of other embodiments. Moreover, various features are described which may be exhibited by some embodiments and not by others.

The use of chest CT when a patient has an infectious disease, such as SARS-CoV-2, may introduce contamination risk to staff and nearby patients during medical procedures, such as imaging. Chest CT can provide valuable information but it often requires 30-90 minutes of CT room decontamination and passive air exchange, which takes a heavy toll on workflow and productivity. For example, the exact decontamination time after CT of a patient with a diagnosis or suspicion for COVID-19 depends upon air exchange rate per hour and passive airflow, ideally in a negative pressure setting. While advanced staff training, dedicated equipment and hallways, and pre-emptive standardized operating procedures may reduce risk to staff, a single infected patient or breach in technique can have profound implications. In an example, in the setting of a pandemic from a droplet-transmitted novel virus and an immune-naïve population, there is a critical clinical need for cost-effective disposable PPE for the infected patient's isolation while undergoing CT or other imaging procedures.

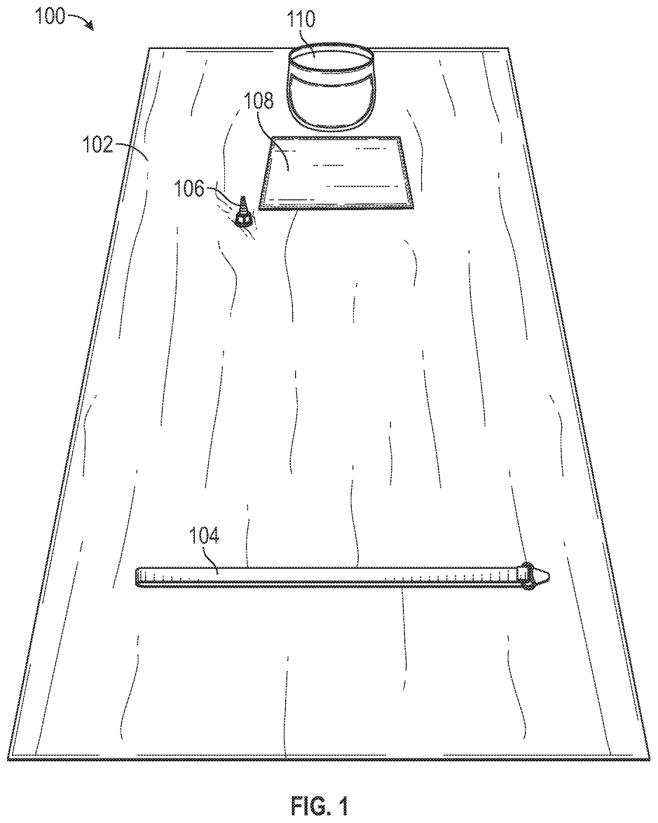

Provided herein is a personal protective patient isolation apparatus. In various embodiments, as shown in FIG. 1, the patient isolation apparatus 100 may include a vapor barrier 102, a fastening strap 104, one or more air inlets 106, and an air filter 108. All components of the patient isolation apparatus may be disposable. The personal protective patient isolation apparatus overcomes the issues described above. For example, the personal protective patient isolation apparatus may be operable to isolate a patient with an infectious disease from medical personnel or other patients while the patient is undergoing a medical procedure or imaging. In some examples, the infectious disease is SARS-CoV-2, Ebola, SARS, MERS, or multi-drug resistant tuberculosis.

Referring to FIGS. 1-3, the vapor barrier 102 may contain or have attached thereto the air filter 108, one or more air inlets 106, and/or the fastening strap 104. The vapor barrier may be impermeable to vapor, such that any gasses, droplets, or aerosols are incapable of traversing the vapor barrier. The vapor barrier may be flexible such that it may surround, conform to, and/or envelop at least the head of the patient. This isolates the patient such that any pathogens exhaled by the patient cannot be transmitted outside the vapor barrier. In some embodiments, the vapor barrier is made of a polymer, such as a thin disposable plastic material and/or an acoustically transparent plastic material. In at least one example, the vapor barrier may be made of a hypoallergenic plastic polymer, such as polyurethane, polyethylene, or CIV-Flex™. The vapor barrier material may reduce or not create artifacts on imaging systems, may reduce the signal to noise ratio, and may not influence the radiation dose to the patient compared to standard isolation chambers. Any more substantial non-disposable device or chamber may also cause more artifacts on CT or MRI and/or may reduce the signal to noise ratio more than the thin disposable plastic material used in the vapor barrier, and may reduce the signal to noise ratio more than the simple low-profile plastic vapor barrier. The vapor barrier does not influence radiation dose to the patient, compared to the isolation chamber, which causes increased radiation (via scatter or dose modulation).

The vapor barrier 102 may have a single opening. In at least one example, the vapor barrier may be a bag. In some embodiments, the vapor barrier is dimensioned to substantially envelop the patient. In various examples, the vapor barrier may have a length of about 50 in to about 60 in and a width of about 25 in to about 40 in. In at least one example, the vapor barrier may be about 57 in by about 33 in. The opening of the vapor barrier may be positioned at or below the patient's waist. In some examples, the vapor barrier is dimensioned to envelop the upper portion of the patient. The upper portion of the patient may include the patient's head and chest, or any portion above the patient's waist. In some examples, the vapor barrier may be dimensioned to extend below the patient's waist and cover at least a portion of the patient's legs.

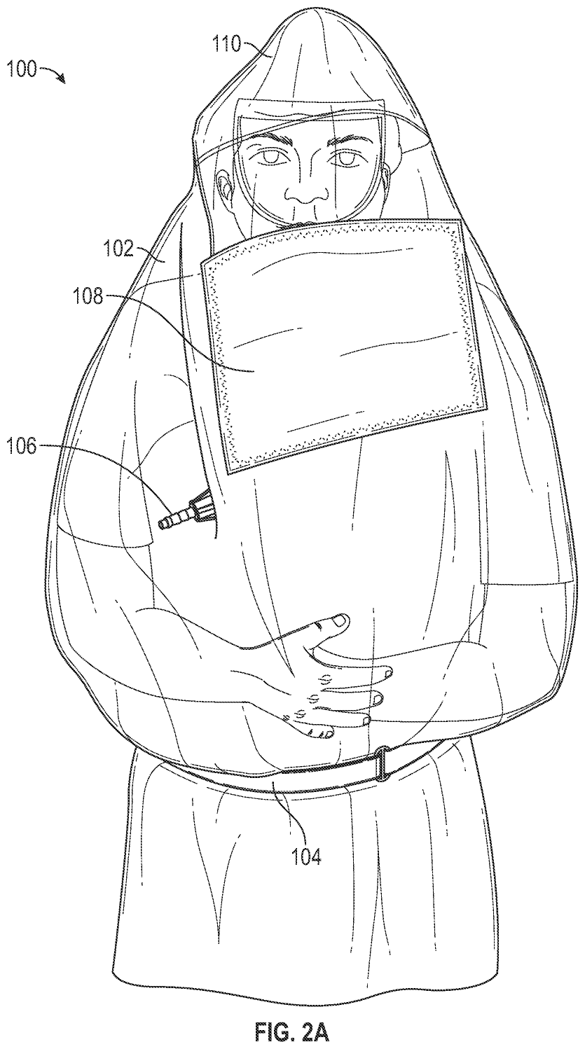

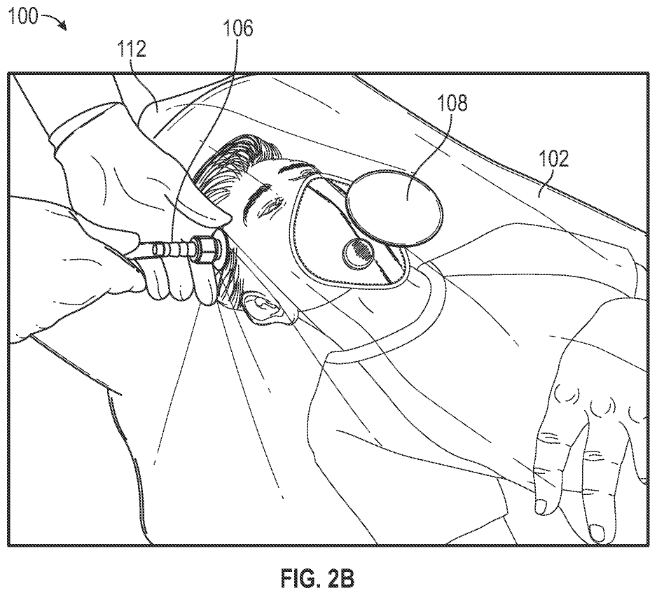

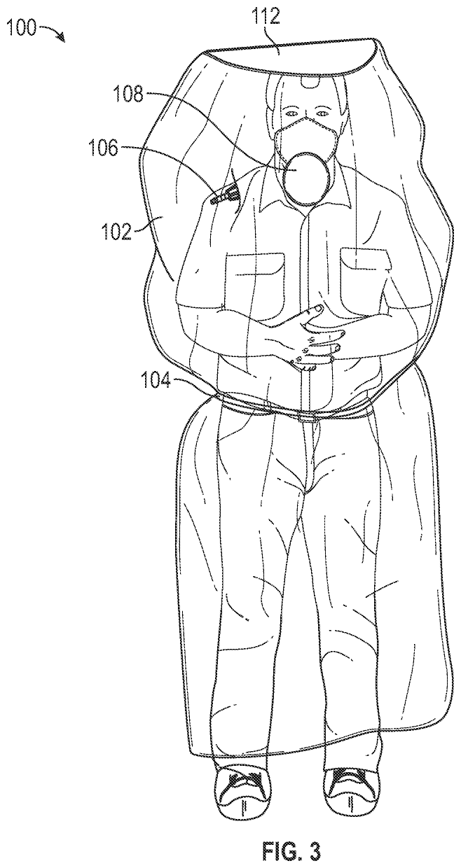

The fastening strap 104 is operable to attach the vapor barrier to a patient. The fastening strap 104 may be connected to the exterior of the vapor barrier 102 and fasten around the patient's waist, thereby fastening the vapor barrier to the patient, as seen in FIGS. 2A and 3. In an embodiment, the fastening strap is provided separate from the vapor barrier such that the fastening strap is not connected to the vapor barrier prior to being used to attach the vapor barrier to the patient. In some examples, the fastening strap may be a wide elastic band or a Velcro belt. The Velcro belt may be a non-elastic belt with a loop for adjustability or may be an elastic belt. The fastening strap may have a length of about 45 in to about 55 in. The fastening strap may be connected to the vapor barrier just proximal to its open end (FIG. 1). The fastening strap may seal the vapor barrier around the head and chest of the patient, forming an interior space (i.e. patient compartment) around the patient. A tight seal is maintained between the patient compartment (enclosing the head and chest) and the outside room air. The fastening strap allows for the patient to be isolated within patient compartment created by the vapor barrier and fastening strap. In some embodiments, the vapor barrier and fastening strap may be configured to provide a hermetic seal.

The one or more air inlets 106 may be integrated with the vapor barrier 102 and sealed along the outside of the vapor barrier 102. The one or more air inlets may be secured to the vapor barrier using one or more washers. For example, a first washer may be on the outside of the vapor barrier and a second washer may be on the inside of the vapor barrier. The one or more washers may be made of rubber or plastic. In an embodiment. The one or more air inlets traverse the vapor barrier to introduce a gas through the vapor barrier and/or suction air from the patient compartment within the vapor barrier. In some examples, the one or more air inlets may be a Christmas tree adapter.

In an embodiment, the air inlet may be an inlet operable to connect to a gas source on the outside of the vapor barrier and a nasal cannula on the interior of the vapor barrier. FIG. 2B shows a patient wearing the patient isolation apparatus 100 laying down and the air inlet 106 being connected to a gas source. The gas from the gas source may include oxygen or other breathing gases. In some examples, the gas may be directly administered to the patient via an integrated nasal cannula to deliver more dedicated oxygen to those patients in need. In other examples, the gas fills the interior space formed by the vapor barrier and the fastening strap around the patient. The amount of gas administered to the patient through the air inlet may be varied depending on the need of the particular patient. In an embodiment, the air inlet is an oxygen exchange nozzle and is connected to an oxygen source with low flow oxygen (0.5 lpm) to avoid aerosolization of infectious droplets, such as might occur with high flow oxygen, or without the vapor barrier.

In some embodiments, the patient isolation apparatus may further include a port (or second air inlet) operable to connect to a portable suction, a negative pressure pump equipped with an in-line HEPA filter suction device, or a healthcare facility wall-mounted suction. The device connected to the port may create negative pressure within the patient compartment formed by the vapor barrier and the fastening belt.

In some embodiments, the air inlet may not be connected to a gas source. For example, the air inlet may be an afferent nozzle with a one-way valve for one-way entry of room air to the inside the vapor barrier.

The air filter 108 is incorporated into the vapor barrier 102 surface to filter air traversing from an interior space (patient compartment) formed by the vapor barrier 102 to an exterior environment. This may include air exhaled by the patient. In some examples, the air filter may be operable to capture one or more pathogens exhaled by the patient. In other examples, the air filter may be operable to capture droplets or aerosols exhaled from the patient. The air filter may have a filtration efficiency ranging from about 90% to 99.7% and an air permeability ranging from about 375-85 CFM. Non-limiting examples of air filters include N-95 filters, KN-95 filters, FFP2 materials, HEPA filters, blended synthetic fiber materials, and spun-bound polypropylene. In an example, the air filter may be a negative flow HEPA filter device. In some embodiments, the patient isolation apparatus may further include a port (or second air inlet) operable to connect to a portable suction or negative pressure pump equipped with an in-line HEPA filter suction device or connect to a healthcare facility wall-mounted suction. Air may flow in to the patient compartment, then slowly through an integrated air filter flush to the vapor barrier (FIG. 2). In another example, a second integrated nozzle for efferent flow may be connected to the air filter sealed around the sub-centimeter nozzle with a tight redundant elastic band.

The air filter 108 may be sized to allow adequate air flow from the patient compartment. In some examples, the air filter may be at least about 5 in by at least about 5 in. In at least one example, the air filter may be about 8 in by about 8 in.

Patients may also be required to wear an N-95/FFP2 mask as an added measure of protection against droplet formation as well as a prophylactic against asphyxiation, by preventing airway obstruction by the vapor barrier material.

The patient isolation apparatus may further include a standoff device to hold the vapor barrier away from the patient. The standoff device may be attached to the interior of the vapor barrier or it may be unattached to the vapor barrier. In some embodiments, the standoff device is in contact with the patient's head. In an example, the standoff device may be a hat or visor 110 worn by the patient. In some embodiments, the patient isolation apparatus 100 may include a disposable plastic visor 110 that sits like an independent cap on the patient's head, under the vapor barrier, to avoid the vapor barrier 102 from falling in the face of the patient (FIGS. 1 and 2A). In another example, the standoff device may be a rigid element 112 attached to the top internal surface of the vapor barrier such that it rests on the patient's head when the patient is enveloped by the vapor barrier. In some examples, the patient isolation apparatus 100 may include a rigid element 112, such as a flat cardboard hat integrated with the vapor barrier 102, to avoid the vapor barrier 102 from falling in the face of the patient (FIG. 3).

Further provided herein is a method for preventing the transmission of an infectious disease from a patient having an airborne infectious disease during a medical procedure. In various embodiments, the method may include covering the patient in a patient isolation apparatus, performing the medical procedure on the patient, and disposing the patient isolation apparatus.

Also provided herein is a method for obtaining medical images of a patient having an airborne infectious disease. The method may include covering the patient in a vapor barrier of a patient isolation apparatus, positioning the patient to obtain the medical images, obtaining the medical images of the patient, and disposing the patient isolation apparatus.

The medical images may be obtained using a computer tomography (CT) apparatus, a magnetic resonance imaging (MRI) apparatus, a positron emission tomography (PET) imaging apparatus and/or any similar radiological imaging system.

The patient may be wearing a NIOSH/EN-approved particulate facepiece respirator or surgical mask whole covered with the patient isolation apparatus.

Covering the patient in the vapor barrier may include supporting each side of the vapor barrier and rolling the vapor barrier over the head first, then carefully rolling the vapor barrier open from the head to waist, without touching the patient. Covering the patient may be done away from the imaging system, such as in a contaminated ante-room or outside. Up to two additional medical professionals may assist in covering the patient, each wearing their own PPE. The method may further include ensuring the vapor barrier is unfolded and is at or below the waist of the patient, inspecting the vapor barrier for any holes or tears, adjusting the vapor barrier such that the air filter is positioned directly in front of the patient's face, and ensuring that the patient's hands are above the patient's waist and within the vapor barrier.

Disposing the vapor barrier may include removing the vapor barrier by lifting the standoff device upwards, without rolling the vapor barrier to minimize exposure to the inner surface of the vapor barrier. Once removed, the vapor barrier may be cinched tight by the fastening strap and air in the vapor barrier may be forced out through the air filter. Disposing of the vapor barrier may be done away from the imaging system, such as in a contaminated ante-room or outside. Disposing the vapor barrier may take place in the same location as the covering step.

The methods may further include connecting a gas source to the air inlet of the patient isolation apparatus. This may further include fitting the patient with a nasal cannula connected to the air inlet. Alternatively, the patient may wear a mask. In some embodiments, the method may further include connecting a suction source or negative pressure pump to a port (or second air inlet) integrated within the vapor barrier of the patient isolation apparatus to provide negative pressure within the patient compartment formed by the vapor barrier and fastening belt.

In addition to the patient during medical imaging procedures, the systems, devices, and methods directed towards the disposable patient isolation apparatus disclosed herein may also be used in various other sedation procedures, elective procedures, bronchoscopy procedures, or during a nebulization treatments in a variety of environments. Additionally, the disposable patient isolation apparatus may be used in any location to sequester patients who may carrier an infectious disease, whether symptomatic or asymptomatic, when isolation beyond mask isolation is desired.

A pandemic has the potential to completely stall radiology department throughput due to excessive delays in between patients for decontamination and airflow exchanges. Broad use of CT has impacted patient isolation in outbreak settings, however only a few patients can be done per shift depending upon air exchanging rates. Using the disposable personal protective isolation apparatus enhances patient safety and staff protection, while avoiding major slowdowns of any infection-specific CT scanner in a cost effective fashion.

The Center for Disease Control (CDC) issues guidelines for patients with infectious diseases such as measles, varicella, pneumonias due to resistant bacteria, and multi-drug resistant tuberculosis, SARS CoV-1, MERS, Ebola, and now COVID-19. Radiology departments traditionally try to accommodate the uncertainties from imaging such patients by performing imaging at the end of the workday, in order to allow for longer air exchange. The CDC has issued guidelines for length of time to allow for passive air exchange after imaging a patient with COVID-19. Whatever the time recommended for passive air exchange, this can become cumbersome and impactful during a pandemic outbreak, when there may be too many patients to let them all wait until the end of the workday, or to wait in between patients.

The CDC recommends a specific time requirement for contaminant removal out to a certain efficiency or percentage (CDC Environmental Infection Control Guidelines, Appendix B: Air, Tables B.1 and B.2 Ancillary/Radiology). The CDC does not however specifically require a negative pressure room for imaging of patients with COVID-19, however the recommendations for delay after such an imaging exam will vary according to the native air exchange rate for the room. As the passive air exchange rate slows down, the delay between patients goes up.

Assuming a 2 hour delay for decontamination and ventilation and a 10 minute fast low-dose scan, a single emergency infectious disease-specific CT scanner might be able to scan about 10 patients in a 24-hour working day. Assuming the patient isolation apparatus enables a fast and safe low-dose chest CT every 10 minutes (with pre-procedure and post procedure preparations taking place next door). This would allow 144 patients to be scanned in the same 24 hour period. This translate into 14.4 times greater patient throughput per day, with the addition of the patient isolation apparatus in a continuously running COVID-19 dedicated CT. Nearly 15 fold enhanced productivity is far greater than any expected cost for a disposable device made from inexpensive and easily-sourced materials.

The CDC COVID-19 guidelines include consideration to provide portable x-ray equipment in patient cohort areas to reduce the need for patient transport. Thus, the patient isolation apparatus may be used for protection during inpatient transport to radiology for CT, as the CDC recommends simple mask coverage during transport. In order to benefit from any added value of CT over chest x-ray, risk reduction during transport may occur with the disposable patient isolation apparatus. Then, after the patient leaves CT, all radiology and environmental cleaning staff may refrain from entering the CT room until sufficient time has elapsed for enough air changes to remove potentially infectious particles.

There are minimal ventilation specifications from the CDC for construction of diagnostic CT rooms. However, best practices may include mitigating risk via extra delays between patients, allowing for more passive air exchange, or alternatively temporary negative pressure isolation via portable anterooms, or plastic curtains with zippers, as commonly used in healthcare facility construction. These may be used in conjunction with the portable isolation bag.

Previous use of containment devices in CT, MRI, and PET have focused on either a high-tech, complex, bulky and expensive high-level containment chamber or isolation pod, or a super low-tech medical waste bag. The former was designed for critical care use or transport in field or military settings, whereas the latter was used to reduce risk in the COVID-19 outbreak in Hubei Province to enhance CT in screening in fever clinics. The ability of such devices to contain infectious agents such as Ebola, SARS, MERS, multi-drug resistant tuberculosis or SARS-CoV-2 requires arresting contact, fomite, droplet, and respiratory aerosols. However, the bulky isolation chamber is not as ergonomic nor conveniently portable as the patient isolation apparatus. SARS-CoV-2 requires such droplet and aerosol precautions, and the patient isolation apparatus may augment patient and staff safety. In some examples, the patient isolation apparatus described herein may have less risk for contaminated air escaping into the CT room or surrounding environment, compared to using simply a standard medical waste bag.

In addition to reducing contamination of imaging rooms and radiology departments, the disposable patient isolation apparatus may meet an urgent clinical need brought about by an unprecedented pandemic. Such a cost-effective device may be useful in any situation where the CT might not reside in a negative pressure setting, which may be common in both inpatient and outpatient imaging centers. Such issues may have added relevance in countries without resources requisite for construction of negative pressure ventilation in radiology or interventional radiology departments. The especially contagious SARS-CoV-2 virus potentially remains viable for several days on surfaces after nebulization, such as may occur in contaminated CT rooms.

### EXAMPLES

Multiple proof-of-concept prototypes were designed, custom fabricated, and test-to-fit on simulated adult and pediatric patients for testing of features intended to minimize droplet spread, while avoiding claustrophobia and risk of asphyxiation. Center for Disease Control (CDC) guidelines were reviewed as relevant to CT decontamination and isolation. A hypoallergenic plastic polymer (CIV-Flex™) was used for the main component skin of the apparatus.

It is important to educate and implement standard operating procedure for all staff and patients well before device implementation. Failures modes must be understood and avoided. A standardized protocol was implemented with extensive staff training in order to avoid contamination of staff or patients due to risky or incorrect doffing or donning processes. The same dedicated space was used for all doffing and donning, ideally a special contaminated ante-room. Two staff optimally assisted, one on each side of an upright or supine patient (FIG. 3). All staff donned their own PPE prior to the patient arrival, including an N-95/FFP2 mask, hat, gown, gloves, shoe covers, and eye protection. Each side was supported as the vapor barrier was rolled over the head first, then carefully rolled open from head to waist, without touching the patient. The CT technologist stayed in the control room as much as possible with verbal cues and visual monitoring from outside the room.

Bag removal (doffing) was accomplished with slow lifting of the bag from the head, by lifting the visor towards the ceiling, without rolling the bag so as to minimize exposure to the inner surface of the bag. This technique minimized contamination from the dirty inside of the bag (which was exposed to droplets) to avoid inner bag touching staff or nearby surfaces. The opening of the bag was cinched tightly by tightening the belt, to seal the contaminated air inside and the air is expressed out the filter exit by slowly squeezing and rolling the bag like a toothpaste tube, from the lower open side towards the head and filter patch. Removal of the bag was done in the same contaminated location, away from the CT scanner, possibly outside, or in a special contaminated ante room nearby. Bag disposal requires a contaminated trash bin, after the air has been squeezed out and completely removed through the filter patch, by staff still wearing N-95 level PPE.

It should be understood from the present disclosure that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A personal protective patient isolation apparatus comprising:
a vapor barrier;
an air filter incorporated within the vapor barrier;
an air inlet traversing the vapor barrier; and
a fastening strap configured to attach the vapor barrier to a patient.

2. The patient isolation apparatus of claim 1, wherein the vapor barrier and the fastening strap are configured to form a patient compartment around the patient.

3. The patient isolation apparatus of claim 2, wherein the vapor barrier and the fastening strap are further configured to provide a hermetic seal.

4. The patient isolation apparatus of claim 1, wherein the vapor barrier is dimensioned to substantially envelop the patient.

5. The patient isolation apparatus of claim 1, further comprising a standoff device to hold the vapor barrier away from the patient.

6. The patient isolation apparatus of claim 5, wherein the standoff device is attached to the vapor barrier.

7. The patient isolation apparatus of claim 5, wherein the standoff device is separate from the vapor barrier and configured to be worn on the patient's head.

8. The patient isolation apparatus of claim 1, wherein the air inlet is configured to allow gas from a gas source to pass through the vapor barrier.

9. The patient isolation apparatus of claim 1, further comprising a port traversing the vapor barrier, the port configured to connect to a suction source to create negative pressure within the vapor barrier.

10. The patient isolation apparatus of claim 1, wherein the air filter is configured to filter air traversing from an interior space formed by the vapor barrier to an exterior environment.

11. The patient isolation apparatus of claim 10, wherein the air filter comprises a N-95 filter, a KN-95 filter, a FFP2 material, a HEPA filter, a blended synthetic fiber material, or spun-bound polypropylene.

12. The patient isolation apparatus of claim 1, wherein the vapor barrier comprises a polymer.

13. The patient isolation apparatus of claim 12, wherein the vapor barrier has reduced artifacts on imaging systems, has reduced signal to noise ratio, and does not influence a radiation dose to the patient compared to standard isolation chambers.

14. A kit comprising:
the patient isolation apparatus of claim 1;
a standoff device not connected to the vapor barrier; and
a nasal cannula.

15. A method for preventing the transmission of an infectious disease from a patient having an airborne infectious disease during a medical procedure, the method comprising:
covering the patient in a patient isolation apparatus, the patient isolation apparatus comprising a vapor barrier, an air inlet, an air filter, and a fastening strap;
performing the medical procedure on the patient; and
disposing the patient isolation apparatus.

16. A method for obtaining medical images of a patient having an airborne infectious disease, the method comprising:
covering the patient in a patient isolation apparatus, the patient isolation apparatus comprising a vapor barrier, an air inlet, an air filter, and a fastening belt;
positioning the patient to obtain the medical images;
obtaining the medical images of the patient; and
disposing the patient isolation apparatus.

17. The method for obtaining medical images of claim 16, wherein the medical images are obtained using a computer tomography (CT) apparatus, a magnetic resonance imaging (MRI) apparatus, or a positron emission tomography (PET) imaging apparatus.

18. The method for obtaining medical images of claim 16, further comprising connecting a gas source to the air inlet of the patient isolation apparatus.

19. The method for obtaining medical images of claim 18, further comprising fitting the patient with a nasal cannula connected to the air inlet.

20. The method for obtaining medical images of claim 16, further comprising connecting a suction source to a port integrated in the vapor barrier.
